(19) United States
(12) Patent Application Publication
(10) Pub. No.: US 2020/0316363 A1
(43) Pub. Date: Oct. 8, 2020

(54) ELECTROTHERAPY APPARATUS, SYSTEM AND METHOD FOR ENHANCED DRUG DELIVERY

(71) Applicant: Quanqin Dai, Diamond BarUS, CA (US)

(72) Inventor: Quanqin Dai, Diamond BarUS, CA (US)

(21) Appl. No.: 16/372,406

(22) Filed: Apr. 2, 2019

(51) Int. Cl. A61N 1/04; A61B 5/00; A61K 9/70

(52) U.S. Cl. CPC A61N 1/0448; A61N 1/325; A61K 9/7038; A61B 5/6833

(57) ABSTRACT

A system and method of electrotherapy for enhanced drug delivery, comprising an electrode patch and an electrotherapy apparatus providing electrical current onto the electrode patch. The electrode patch contains one or more active drug ingredients in a layer of conductive gel, and the electrotherapy apparatus provides electrical current onto the electrode patch for the enhanced drug delivery of active drug ingredients. The conductive gel is in contact with a human or animal body, and the electrode patch also includes a layer of conductive sheet, a supporting layer, and conductive connectors. The conductive gel has the viscosity allowing it to adhere to skin on a human or animal body. The active drug ingredients that can be delivered through the conductive gel include: lidocaine, capsaicin, menthol, camphor, methyl salicylate, trolamine salicylate, Arnica montana, Calendula officinalis, Hypericum perforatum, Symphytum officinale, cannabidiol (CBD) or any combination of these substances. The mass fraction of the active drug ingredients can vary from 0.001% to 30%. The electrotherapy device can be reused.

### BACKGROUND

The transdermal route ranks as the successful innovative research area in drug delivery, and about 40% of the drug delivery candidate products under clinical evaluation are related to the transdermal or dermal system. It is reported the worldwide market of transdermal patches approaches £2 billion. To achieve the desired therapeutic effect, the success of a dermatological drug for the drug delivery really depends on its ability to penetrate through skin in sufficient quantities. With the currently available format and design, only 2-3% of a typical lidocaine patch containing 700 mg of lidocaine is delivered. It means at least 97% of the dose is disposed of, which generates significant waste and hazard.

### SUMMARY OF INVENTION

An enhanced system comprises an electrode patch and an electrotherapy apparatus. The electrode patch contains the active drug ingredient in the layer of conductive gel for attaching the patch onto the human or animal body. The electrotherapy apparatus provides the electrical current onto the electrode patch for enhancing the release of the active drug ingredient into the human or animal body. This invention has the function of enhancing the drug delivery and increasing the blood circulation by applying the electrotherapy. The electrode patch containing the above described active drug ingredient can be used alone, and also be used with the electrotherapy apparatus, offering a wide range of application. In addition, the electrotherapy apparatus in this invention could be reused, greatly reducing the user's economic burden.

### TECHNICAL FIELD

The present invention relates to an enhanced drug delivery, and more particularly to an electrotherapy for the enhanced drug delivery.

### BRIEF DESCRIPTION OF THE DRAWINGS

FIG. 1 is a view of a first embodiment of the present invention.

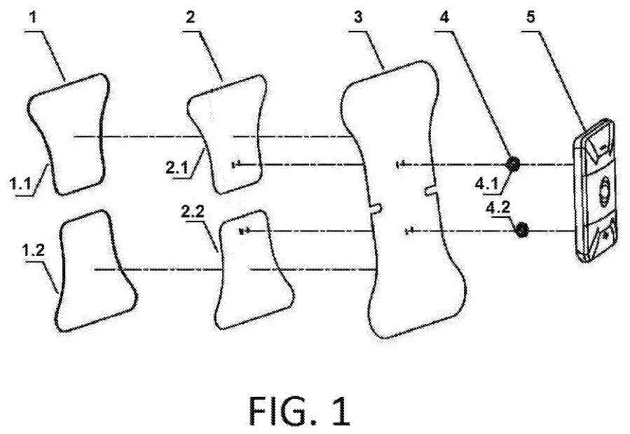

FIG. 2 is a view of a second embodiment of the present invention.

FIG. 3 is a view of a third embodiment of the present invention.

FIG. 4 is a view of a fourth embodiment of the present invention.

FIG. 5 is a view of a fifth embodiment of the present invention.

### DETAILED DESCRIPTION

It is an object of the present invention to provide an electrotherapy system for the enhanced drug delivery, which is simple, economical, effective, and wide application to overcome deficiencies in the prior art.

An enhanced system comprises an electrode patch and an electrotherapy apparatus. The electrode patch contains the active drug ingredient in the layer of conductive gel for attaching the patch onto the human or animal body. The electrotherapy apparatus provides the electrical current onto the electrode patch for enhancing the release of the active drug ingredient into the human or animal body.

The layer of conductive gel with the active drug ingredient contained is in contact with the human or animal body. The active drug ingredient contained in the conductive gel is lidocaine, capsaicin, menthol, camphor, methyl salicylate, trolamine salicylate, Arnica montana, Calendula officinalis, Hypericum perforatum, Symphytum officinale, cannabidiol (CBD), and/or any combination of these substances, the mass fraction of which is from 0.001% to 30%. The layer of conductive gel is divided into at least the first conductive gel area and the second conductive gel.

In addition to the layer of conductive gel, the electrode patch also includes a layer of conductive sheet, a supporting layer, and conductive connector.

The layer of conductive sheet is separated into at least the first conductive sheet for the first conductive gel and the second conductive sheet for the second conductive gel.

The layer of conductive sheet is mounted onto the supporting layer, on which the conductive connector is located. Alternatively, the conductive connector is connected to a lead wire that is located between the layer of conductive sheet and the supporting layer.

Through the conductive connector, the electrode patch with the active drug ingredient contained is electrically connected to the electrotherapy apparatus, significantly enhancing the drug delivery. The electrode patch can be used alone, and also be used with the electrotherapy apparatus, offering a wide range of application. In addition, the electrotherapy apparatus in this invention could be reused, greatly reducing the user's economic burden.

### The First Embodiment

The first embodiment of the present invention comprises a layer of conductive gel 1, a layer of conductive sheet 2, a supporting layer 3, conductive connector 4, and an electrotherapy apparatus 5. The layer of conductive gel 1 with the active drug ingredient contained is divided into at least the first conductive gel 1.1 and the second conductive gel 1.2. The layer of conductive sheet 2 is separated into at least the first conductive sheet 2.1 for the first conductive gel 1.1 and the second conductive sheet 2.2 for the second conductive gel 1.2. The first conductive sheet 2.1 is connected to the first conductive connector 4.1 and the second conductive sheet 2.2 is connected to the second conductive connector 4.2. Each of the first conductive connector 4.1 and the second conductive connector 4.2 is located on the supporting layer 3 or between the layer of conductive sheet 2 and the supporting layer 3. The layer of conductive gel 1, the layer of conductive sheet 2, and the supporting layer 3 are sequentially mounted onto each other. The layer of conductive gel 1 has a certain viscosity so it could adhere to the human or animal body. The electrode patch containing the above described active drug ingredient can be used alone, and can also be used with the electrotherapy apparatus. With the electrotherapy apparatus applied, the drug delivery from the electrode patch to the human or animal body could be increased by many times.

Further, the layer of conductive gel 1 comprises the following active drug ingredients: lidocaine, capsaicin, menthol, camphor, methyl salicylate, trolamine salicylate, Arnica montana, Calendula officinalis, Hypericum perforatum, Symphytum officinale, cannabidiol (CBD), and/or any combination of these substances, the mass fraction of which is from 0.001% to 30%. In the process of making the conductive gel 1, it is necessary to carry out the steps of feeding, mixing and/or coating, which can be completed by automatic production line to ensure the quality and consistency of the conductive gel. Afterward, the conductive gel 1 is cut into the appropriate size/shape and attached to the conductive sheet for making the electrode patch.

Further, the layer of conductive sheet 2 is attached on one side of the supporting layer 3, and the other side of the supporting layer 3 is equipped with two conductive connectors 4.1 and 4.2 which are electrically connected to the layer of conductive sheet 2. The two conductive connectors 4.1 and 4.2 on the supporting layer 3 are electrically connected to the electrotherapy apparatus 5. The connection between the conductive connector 4 and the electrotherapy apparatus 5 includes the mechanical fastening, magnetic contact, plug, and the like.

### The Second Embodiment

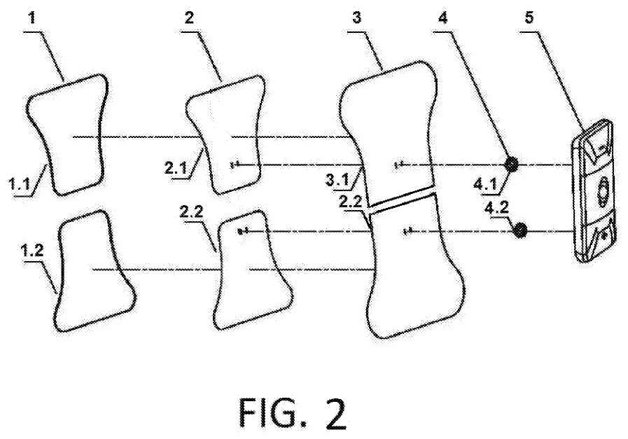

The second embodiment is referred to in FIG. 2. Its difference from the first embodiment is as follows: the supporting layer 3 is separated into at least the first supporting layer 3.1 for the first conductive sheet 2.1 and the second supporting layer 3.2 for the second conductive sheet 2.2. Two pieces or an even number of pieces of such electrode patch are used together with the electrotherapy apparatus 5 through the two conductive connectors 4.1 and 4.2.

The other parts of the second embodiment are the same as the first embodiment, and will not be described here.

### The Third Embodiment

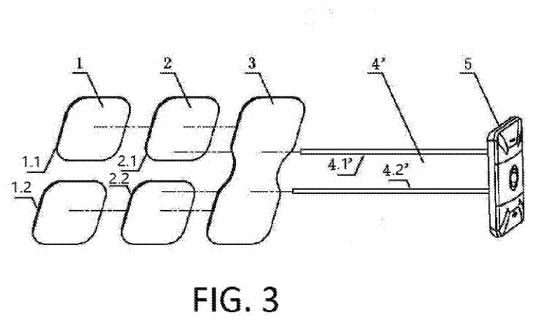

The third embodiment is referred to in FIG. 3. Its difference from the first embodiment is the electrotherapy apparatus 5 is connected to the conductive connector of the lead wire 4′. The lead wire 4′ is separated into at least the first lead wire 4.1′ and the second lead wire 4.2′.

The other parts of the third embodiment are described in the first embodiment, and thereof will not be described here.

### The Fourth Embodiment

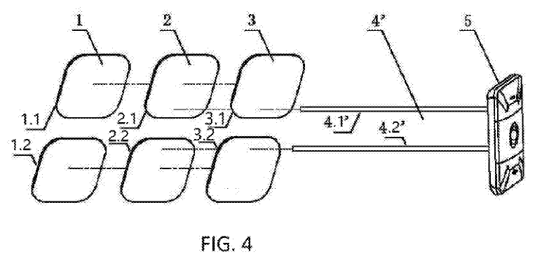

The fourth embodiment is referred to in FIG. 4. Its difference from the first embodiment is as follows: The supporting layer 3 is separated into at least the first supporting layer 3.1 for the first conductive sheet 2.1 and the second supporting layer 3.2 for the second conductive sheet 2.2. Two pieces or an even number of pieces of such electrode patch are used together with the electrotherapy apparatus 5 through the conductive connector of lead wire 4′. The lead wire 4′ is separated into at least the first lead wire 4.1′ and the second lead wire 4.2′.

The other parts of the fourth embodiment are described in the first embodiment, and thereof will not be described here.

### The Fifth Embodiment

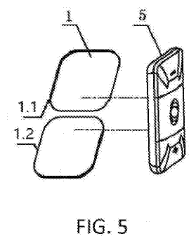

The fifth embodiment, is referred to FIG. 5. It differs from the first embodiment as follows: The supporting layer 3 is shared with the electrotherapy apparatus 5; the conductive sheet 2 is provided with two pieces or an even number of pieces and is directly placed on the electrotherapy apparatus 5, so the conductive connector 4 are not shown; the two pieces of conductive gel 1.1 and 1.2 are directly attached to the two pieces of conductive sheet 2.1 and 2.2 on the electrotherapy apparatus 5.

The other parts of the fifth embodiment are described in the first embodiment, and thereof will not be described here.

### The Sixth Embodiment

The sixth embodiment differs from the first embodiment as follows: there is a computerized control system for the purpose of regulating the release of the active drug ingredients, wherein this computerized control system can be programmed for the purpose of the optimal method to release any or a combination of the active drug ingredients, and can optionally connect to the internet in order to continuously receive directions on how to regulate the release of the active drug ingredients.

### The Seventh Embodiment

The seventh embodiment differs from the first embodiment as follows: the power source for the electrotherapy apparatus is portable solar power device or other portable power device that can be worn by a human or animal, or attached to clothing, and the rest of the invention can also be worn or attached to clothing, such that the entire invention can be portable and utilized by a human or animal in a hands-free manner.

### The Eighth Embodiment

The eighth embodiment differs from the first embodiment as follows: an electrotherapy apparatus includes the capabilities of monitoring and controlling the voltage associated with the patient's use of the electrotherapy apparatus, as well as starting and stopping the enhancement of the active drug ingredients.

### The Ninth Embodiment

The ninth embodiment differs from the first embodiment as follows: There is a computerized control system for the purpose of regulating the release of the active drug ingredients, wherein this computerized control system can be programmed for the purpose of the optimal method to release any or a combination of the active drug ingredients, and can optionally connect to the internet in order to continuously receive directions on how to regulate the release of the active drug ingredients. The power source for the electrotherapy apparatus is portable solar power device or other portable power device that can be worn by a human or animal, or attached to clothing, and the rest of the invention can also be worn or attached to clothing, such that the entire invention can be portable and utilized by a human or animal in a hands-free manner. An electrotherapy apparatus includes the capabilities of monitoring and controlling the voltage associated with the patient's use of the electrotherapy apparatus, as well as starting and stopping the enhancement of the active drug ingredients.

### The Tenth Embodiment

The tenth embodiment of the present invention is a system of electrotherapy for enhanced drug delivery, comprising an electrode patch containing one or more active drug ingredients in a layer of at least a first conductive gel and a second conductive gel; an electrotherapy apparatus that provides electrical current onto the electrode patch; wherein the layer of the first conductive gel and the second conductive gel is in contact with a human or animal body; wherein the electrode patch also includes a layer of at least a first conductive sheet and a second conductive sheet, a supporting layer, and at least a first conductive connector and a second conductive connector; wherein the first conductive sheet is connected to the first conductive gel; wherein the second conductive sheet is connected to the second conductive gel; and wherein the first conductive gel and the second conductive gel have a viscosity allowing both gels to adhere to skin on a human or animal body.

### The Eleventh Embodiment

The eleventh embodiment differs from the tenth embodiment as follows: the active drug ingredients can be lidocaine, capsaicin, menthol, camphor, methyl salicylate, trolamine salicylate, Arnica montana, Calendula officinalis, Hypericum perforatum, Symphytum officinale, cannabidiol (CBD) or any combination of these substances; wherein the mass fraction of the active drug ingredients is from 0.001% to 30%.

### The Twelfth Embodiment

The twelfth embodiment differs from the tenth embodiment as follows: Wherein the layer of the first conductive sheet and the second conductive sheet is mounted onto the supporting layer, and the first conductive sheet is connected to the first conductive connector and the second conductive sheet is connected to the second conductive connector.

### The Thirteenth Embodiment

The thirteenth embodiment differs from the tenth embodiment as follows: each of the first conductive connector and the second conductive connector is located on the supporting layer or between the layer of conductive sheet and the supporting layer.

### The Fourteenth Embodiment

The fourteenth embodiment differs from the tenth embodiment as follows: the electrode patch containing the active drug ingredients is electrically connected to the electrotherapy apparatus through the first conductive connector and the second conductive connector in order to enhance the drug delivery of the active drug ingredients; and the electrotherapy apparatus can be reused.

### The Fifteenth Embodiment

The fifteenth embodiment is as follows: a system of electrotherapy for enhanced drug delivery, comprising at least two electrode patches, each of which contains one or more active drug ingredients in a layer of a conductive gel; an electrotherapy apparatus that provides electrical current onto the two electrode patches; wherein the layer of conductive gel is in contact with a human or animal body, wherein each electrode patch also includes a layer of a conductive sheet, a supporting layer, and a conductive connector; wherein the conductive sheet is connected to the conductive gel; and wherein the conductive gel has a viscosity allowing gel to adhere to skin on a human or animal body.

### The Sixteenth Embodiment

The sixteenth embodiment differs from the fifteenth embodiment as follows: the active drug ingredients can be lidocaine, capsaicin, menthol, camphor, methyl salicylate, trolamine salicylate, Arnica montana, Calendula officinalis, Hypericum perforatum, Symphytum officinale, cannabidiol (CBD) or any combination of these substances; wherein the mass fraction of the active drug ingredients is from 0.001% to 30° %.

### The Seventeenth Embodiment

The seventeenth embodiment differs from the fifteenth embodiment as follows: the layer of conductive sheet is mounted onto the supporting layer, and the conductive sheet is connected to the conductive connector.

### The Eighteenth Embodiment

The eighteenth embodiment differs from the fifteenth embodiment as follows: the conductive connector is located on the supporting layer or between the layer of conductive sheet and the supporting layer.

### The Nineteenth Embodiment

The nineteenth embodiment differs from the fifteenth embodiment as follows: each of the two electrode patches containing the active drug ingredients is electrically connected to the electrotherapy apparatus through the conductive connector in order to enhance the drug delivery of the active drug ingredients; and the electrotherapy apparatus can be reused.

### The Twentieth Embodiment

The twentieth embodiment is another embodiment of the present invention, it is as follows: a system of electrotherapy for enhanced drug delivery, comprising a layer of at least a first conductive gel and a second conductive gel, each of which contains one or more active drug ingredients; an electrotherapy apparatus that provides electrical current onto a layer of at least a first conductive sheet and a second conductive sheet; wherein the first conductive sheet and the second conductive sheet is directly placed on the electrotherapy apparatus; wherein the first conductive sheet is connected to the first conductive gel and the second conductive sheet is connected to the second conductive gel; wherein the first conductive gel and the second conductive gel are in contact with a human or animal body; and wherein the first conductive gel and the second conductive gel have a viscosity allowing both gels to adhere to skin on a human or animal body.

### The Twenty-First Embodiment

The twenty-first embodiment differs from the twentieth embodiment as follows: the active drug ingredients can be lidocaine, capsaicin, menthol, camphor, methyl salicylate, trolamine salicylate, Arnica montana, Calendula officinalis, Hypericum perforatum, Symphytum officinale, cannabidiol (CBD) or any combination of these substances; wherein the mass fraction of the active drug ingredients is from 0.001% to 30%.

### The Twenty-Second Embodiment

The twenty-second embodiment differs from the twentieth embodiment as follows: the first conductive gel and the second conductive gel containing the active drug ingredients are electrically connected to the electrotherapy apparatus through the first conductive sheet and the second conductive sheet in order to enhance the drug delivery of the active drug ingredients; and the electrotherapy apparatus can be reused.

### The Twenty-Third Embodiment

The twenty-third embodiment is another embodiment of the present invention, it is as follows: a system of electrotherapy for enhanced drug delivery, comprising an electrode patch containing one or more active drug ingredients in a layer of at least a first conductive gel and a second conductive gel; an electrotherapy apparatus that provides electrical current onto the electrode patch; wherein the layer of the first conductive gel and the second conductive gel is in contact with a human or animal body; wherein the electrode patch also includes a layer of at least a first conductive sheet and a second conductive sheet, a supporting layer, and at least a first conductive connector and a second conductive connector; wherein the first conductive sheet is connected to the first conductive gel; wherein the second conductive sheet is connected to the second conductive gel; and wherein the first conductive gel and the second conductive gel have a viscosity allowing both gels to adhere to skin on a human or animal body.

The active drug ingredients can be lidocaine, capsaicin, menthol, camphor, methyl salicylate, trolamine salicylate, Arnica montana, Calendula officinalis, Hypericum perforatum, Symphytum officinale, cannabidiol (CBD) or any combination of these substances; wherein the mass fraction of the active drug ingredients is from 0.001% to 30%.

The layer of the first conductive sheet and the second conductive sheet is mounted onto the supporting layer, and the first conductive sheet is connected to the first conductive connector and the second conductive sheet is connected to the second conductive connector.

The electrode patch containing the active drug ingredients is electrically connected to the electrotherapy apparatus through the first conductive connector and the second conductive connector in order to enhance the drug delivery of the active drug ingredients; and the electrotherapy apparatus can be reused.

### The Twenty-Fourth Embodiment

The twenty-fourth embodiment is another embodiment of the present invention, it is as follows: a system of electrotherapy for enhanced drug delivery, comprising at least two electrode patches, each of which contains one or more active drug ingredients in a layer of a conductive gel; an electrotherapy apparatus that provides electrical current onto the two electrode patches; wherein the layer of conductive gel is in contact with a human or animal body; wherein each electrode patch also includes a layer of a conductive sheet, a supporting layer, and a conductive connector; wherein the conductive sheet is connected to the conductive gel; and wherein the conductive gel has a viscosity allowing gel to adhere to skin on a human or animal body.

The active drug ingredients can be lidocaine, capsaicin, menthol, camphor, methyl salicylate, trolamine salicylate, Arnica montana, Calendula officinalis, Hypericum perforatum, Symphytum officinale, cannabidiol (CBD) or any combination of these substances; wherein the mass fraction of the active drug ingredients is from 0.001% to 30%.

The layer of conductive sheet is mounted onto the supporting layer, and the conductive sheet is connected to the conductive connector. The conductive connector is located on the supporting layer or between the layer of conductive sheet and the supporting layer. Each of the two electrode patches containing the active drug ingredients is electrically connected to the electrotherapy apparatus through the conductive connector in order to enhance the drug delivery of the active drug ingredients; and the electrotherapy apparatus can be reused.

### The Twenty-Fifth Embodiment

The twenty-fifth embodiment is another embodiment of the present invention, it is as follows: a system of electrotherapy for enhanced drug delivery, comprising a layer of at least a first conductive gel and a second conductive gel, each of which contains one or more active drug ingredients; an electrotherapy apparatus that provides electrical current onto a layer of at least a first conductive sheet and a second conductive sheet; wherein the first conductive sheet and the second conductive sheet is directly placed on the electrotherapy apparatus; wherein the first conductive sheet is connected to the first conductive gel and the second conductive sheet is connected to the second conductive gel; wherein the first conductive gel and the second conductive gel are in contact with a human or animal body; and wherein the first conductive gel and the second conductive gel have a viscosity allowing both gels to adhere to skin on a human or animal body.

The active drug ingredients can be lidocaine, capsaicin, menthol, camphor, methyl salicylate, trolamine salicylate, Arnica montana, Calendula officinalis, Hypericum perforatum, Symphytum officinale, cannabidiol (CBD) or any combination of these substances; wherein the mass fraction of the active drug ingredients is from 0.001% to 30%.

The first conductive gel and the second conductive gel containing the active drug ingredients are electrically connected to the electrotherapy apparatus through the first conductive sheet and the second conductive sheet in order to enhance the drug delivery of the active drug ingredients; and the electrotherapy apparatus can be reused.

Having described the invention by the description and illustrations above, it should be understood that these are exemplary of the invention and are not to be considered as limiting. Accordingly, the invention is not to be considered as limited by the foregoing description, but includes any equivalents.

What is claimed is:

1. A system of electrotherapy for enhanced drug delivery, comprising
an electrode patch containing one or more active drug ingredients in a layer of at least a first conductive gel and a second conductive gel;
an electrotherapy apparatus that provides electrical current onto the electrode patch;
wherein the layer of the first conductive gel and the second conductive gel is in contact with a human or animal body;
wherein the electrode patch also includes a layer of at least a first conductive sheet and a second conductive sheet, a supporting layer, and at least a first conductive connector and a second conductive connector;
wherein the first conductive sheet is connected to the first conductive gel;
wherein the second conductive sheet is connected to the second conductive gel;
and wherein the first conductive gel and the second conductive gel have a viscosity allowing both gels to adhere to skin on a human or animal body.

2. The system of claim 1, further comprising:
wherein the active drug ingredients can be lidocaine, capsaicin, menthol, camphor, methyl salicylate, trolamine salicylate, Arnica montana, Calendula officinalis, Hypericum perforatum, Symphytum officinale, cannabidiol (CBD) or any combination of these substances;
wherein the mass fraction of the active drug ingredients is from 0.001% to 30%.

3. The system of claim 1, further comprising:
wherein the layer of the first conductive sheet and the second conductive sheet is mounted onto the supporting layer, and the first conductive sheet is connected to the first conductive connector and the second conductive sheet is connected to the second conductive connector.

4. The system of claim 1, further comprising:
wherein each of the first conductive connector and the second conductive connector is located on the supporting layer or between the layer of conductive sheet and the supporting layer.

5. The system of claim 1, further comprising:
wherein the electrode patch containing the active drug ingredients is electrically connected to the electrotherapy apparatus through the first conductive connector and the second conductive connector in order to enhance the drug delivery of the active drug ingredients;
wherein the electrotherapy apparatus can be reused.

6. A system of electrotherapy for enhanced drug delivery, comprising
at least two electrode patches, each of which contains one or more active drug ingredients in a layer of a conductive gel;
an electrotherapy apparatus that provides electrical current onto the two electrode patches;
wherein the layer of conductive gel is in contact with a human or animal body;
wherein each electrode patch also includes a layer of a conductive sheet, a supporting layer, and a conductive connector;
wherein the conductive sheet is connected to the conductive gel;
and wherein the conductive gel has a viscosity allowing gel to adhere to skin on a human or animal body.

7. The system of claim 6, further comprising:
wherein the active drug ingredients can be lidocaine, capsaicin, menthol, camphor, methyl salicylate, trolamine salicylate, Arnica montana, Calendula officinalis, Hypericum perforatum, Symphytum officinale, cannabidiol (CBD) or any combination of these substances;
wherein the mass fraction of the active drug ingredients is from 0.001% to 30%.

8. The system of claim 6, further comprising:
wherein the layer of conductive sheet is mounted onto the supporting layer, and the conductive sheet is connected to the conductive connector.

9. The system of claim 6, further comprising:
wherein the conductive connector is located on the supporting layer or between the layer of conductive sheet and the supporting layer.

10. The system of claim 6, further comprising:
wherein each of the two electrode patches containing the active drug ingredients is electrically connected to the electrotherapy apparatus through the conductive connector in order to enhance the drug delivery of the active drug ingredients;
wherein the electrotherapy apparatus can be reused.

11. A system of electrotherapy for enhanced drug delivery, comprising
A layer of at least a first conductive gel and a second conductive gel, each of which contains one or more active drug ingredients;
an electrotherapy apparatus that provides electrical current onto a layer of at least a first conductive sheet and a second conductive sheet;
wherein the first conductive sheet and the second conductive sheet is directly placed on the electrotherapy apparatus;
wherein the first conductive sheet is connected to the first conductive gel and the second conductive sheet is connected to the second conductive gel;
wherein the first conductive gel and the second conductive gel are in contact with a human or animal body;
and wherein the first conductive gel and the second conductive gel have a viscosity allowing both gels to adhere to skin on a human or animal body.

12. The system of claim 11, further comprising:
wherein the active drug ingredients can be lidocaine, capsaicin, menthol, camphor, methyl salicylate, trolamine salicylate, Arnica montana, Calendula officinalis, Hypericum perforatum, Symphytum officinale, cannabidiol (CBD) or any combination of these substances;
wherein the mass fraction of the active drug ingredients is from 0.001% to 30%.

13. The system of claim 11, further comprising:
wherein the first conductive gel and the second conductive gel containing the active drug ingredients are electrically connected to the electrotherapy apparatus through the first conductive sheet and the second conductive sheet in order to enhance the drug delivery of the active drug ingredients;
wherein the electrotherapy apparatus can be reused.

14. A method of electrotherapy for enhanced drug delivery, comprising
an electrode patch containing one or more active drug ingredients in a layer of at least a first conductive gel and a second conductive gel;
an electrotherapy apparatus that provides electrical current onto the electrode patch;
wherein the layer of the first conductive gel and the second conductive gel is in contact with a human or animal body;
wherein the electrode patch also includes a layer of at least a first conductive sheet and a second conductive sheet, a supporting layer, and at least a first conductive connector and a second conductive connector;
wherein the first conductive sheet is connected to the first conductive gel;
wherein the second conductive sheet is connected to the second conductive gel;
and wherein the first conductive gel and the second conductive gel have a viscosity allowing both gels to adhere to skin on a human or animal body.

15. The method of claim 14, further comprising:
wherein the active drug ingredients can be lidocaine, capsaicin, menthol, camphor, methyl salicylate, trolamine salicylate, Arnica montana, Calendula officinalis, Hypericum perforatum, Symphytum officinale, cannabidiol (CBD) or any combination of these substances;
wherein the mass fraction of the active drug ingredients is from 0.001% to 30%.

16. The method of claim 14, further comprising:
wherein the layer of the first conductive sheet and the second conductive sheet is mounted onto the supporting layer, and the first conductive sheet is connected to the first conductive connector and the second conductive sheet is connected to the second conductive connector.

17. The method of claim 14, further comprising:
wherein each of the first conductive connector and the second conductive connector is located on the supporting layer or between the layer of conductive sheet and the supporting layer.

18. The method of claim 14, further comprising:
wherein the electrode patch containing the active drug ingredients is electrically connected to the electrotherapy apparatus through the first conductive connector and the second conductive connector in order to enhance the drug delivery of the active drug ingredients;
wherein the electrotherapy apparatus can be reused.

19. The method of claim 14, further comprising:
wherein the active drug ingredients can be lidocaine, capsaicin, menthol, camphor, methyl salicylate, trolamine salicylate, Arnica montana, Calendula officinalis, Hypericum perforatum, Symphytum officinale, cannabidiol (CBD) or any combination of these substances;
wherein the mass fraction of the active drug ingredients is from 0.001% to 30%;
wherein the layer of the first conductive sheet and the second conductive sheet is mounted onto the supporting layer, and the first conductive sheet is connected to the first conductive connector and the second conductive sheet is connected to the second conductive connector;
wherein each of the first conductive connector and the second conductive connector is located on the supporting layer or between the layer of conductive sheet and the supporting layer;
wherein the electrode patch containing the active drug ingredients is electrically connected to the electrotherapy apparatus through the first conductive connector and the second conductive connector in order to enhance the drug delivery of the active drug ingredients;
wherein the electrotherapy apparatus can be reused;
wherein the electrotherapy apparatus includes the capabilities of monitoring and controlling the voltage associated with a patient's use of the electrotherapy apparatus;
wherein the electrotherapy apparatus includes the capabilities of starting and stopping the enhancement of the active drug ingredients.
